(19) United States
(12) Patent Application Publication
(10) Pub. No.: US 2021/0193329 A1
(43) Pub. Date: Jun. 24, 2021

(54) MEDICAL INFORMATION PROCESSING SYSTEM, MEDICAL INFORMATION PROCESSING APPARATUS, AND MEDICAL INFORMATION PROCESSING METHOD

(71) Applicant: CANON MEDICAL SYSTEMS CORPORATION, Otawara-shi (JP)

(72) Inventor: Nao WARIISHI, Otawara (JP)

(73) Assignee: CANON MEDICAL SYSTEMS CORPORATION, Otawara-shi (JP)

(21) Appl. No.: 17/132,463

(22) Filed: Dec. 23, 2020

(30) Foreign Application Priority Data
Dec. 24, 2019 (JP) 2019-232692

(51) Int. Cl. G16H 50/70; G16H 70/20; G16H 15/00; G16H 40/20; G16H 10/60; G06F 16/2458; G06F 16/2455; G06F 16/248; A61B 5/00

(52) U.S. Cl. CPC G16H 50/70; G16H 70/20; G16H 15/00; G16H 40/20; G16H 10/60; G08B 21/18; G06F 16/24564; G06F 16/248; A61B 5/746; A61B 5/742; A61B 5/7282; G06F 16/2477

(57) ABSTRACT

A medical information processing apparatus according to an embodiment comprises processing circuitry. The processing circuitry is configured to judge whether or not first clinical information matching an alert issuance reference rule is present; to issue an alert when having determined that the first clinical information matching the alert issuance reference rule is present; to obtain second clinical information relevant to the first clinical information from during a time period preceding a reference point in time at which the first clinical information matching the alert issuance reference rule occurred; and to cause display circuitry to display the second clinical information.

### CROSS-REFERENCE TO RELATED APPLICATIONS

This application is based upon and claims the benefit of priority from Japanese Patent Application No. 2019-232692, filed on Dec. 24, 2019; the entire contents of which are incorporated herein by reference.

### FIELD

Embodiments described herein relate generally to a medical information processing system, a medical information processing apparatus, and a medical information processing method.

### BACKGROUND

Conventionally, a function is known by which specific clinical information is monitored according to a rule set by a user, so as to display an alert on a screen, for example, when a certain medical examination value in the clinical information becomes equal to or higher than a set value and to display data represented by the clinical information matching the rule with an emphasis. By using this function, it is possible to reduce the possibilities where the user may overlook abnormality values or changes in the clinical information.

By using the function described above, the user is able to obtain information indicating which rule was executed and at which point in time the data matched the rule; however, to obtain information from around the time at which the data matching the rule is present, it is necessary to separately access various types of clinical information and to view the data from before and after the time in question.

For example, when an alert is issued about data at a certain time, the user observes the data for which the alert is issued. After that, to understand the cause of the issued alert, the user would need to observe, while using the alerted data as a reference, the data from before and after that time, with respect to other various types of clinical information. In that situation, it would be necessary to manually search for the cause from a huge amount of data including medication administration data and vital sign data. According to the conventional technique, however, multiple human operations would be required to understand the cause of the alert being issued, which would impose a large work burden on the user.

### BRIEF DESCRIPTION OF THE DRAWINGS

FIG. 1 is a diagram illustrating an example of a medical information processing system including a server apparatus, a client apparatus, and a clinical information database;

FIG. 2 is a diagram illustrating an exemplary configuration of the server apparatus serving as a medical information processing apparatus;

FIG. 3 is a diagram illustrating an exemplary configuration of the client apparatus serving as a medical information processing apparatus;

FIG. 4 is a flowchart illustrating an example of a flow in an alert relevant event searching process performed in the medical information processing system;

FIG. 5 is a drawing illustrating an example in which first clinical information is displayed by a display circuit;

FIG. 6 is a drawing illustrating an example in which second clinical information is displayed by the display circuit;

FIG. 7 is a drawing illustrating an example of a search criteria editing screen;

FIG. 8 is a drawing illustrating an example of a screen used for setting priority levels for displaying clinical information;

FIG. 9 is a drawing for explaining an example of a screen used for displaying the second clinical information when it is possible to set display priority levels; and

FIG. 10 is a drawing for explaining another example of a screen used for displaying the second clinical information when it is possible to set display priority levels.

### DETAILED DESCRIPTION

A medical information processing apparatus according to an embodiment comprises processing circuitry. The processing circuitry is configured to judge whether or not first clinical information matching an alert issuance reference rule is present; to issue an alert when having determined that the first clinical information matching the alert issuance reference rule is present; to obtain second clinical information relevant to the first clinical information from during a time period preceding a reference point in time at which the first clinical information matching the alert issuance reference rule occurred; and to cause display circuitry to display the second clinical information.

Exemplary embodiments of a medical information processing system, a medical information processing apparatus, and a medical information processing method will be explained below, with reference to the accompanying drawings.

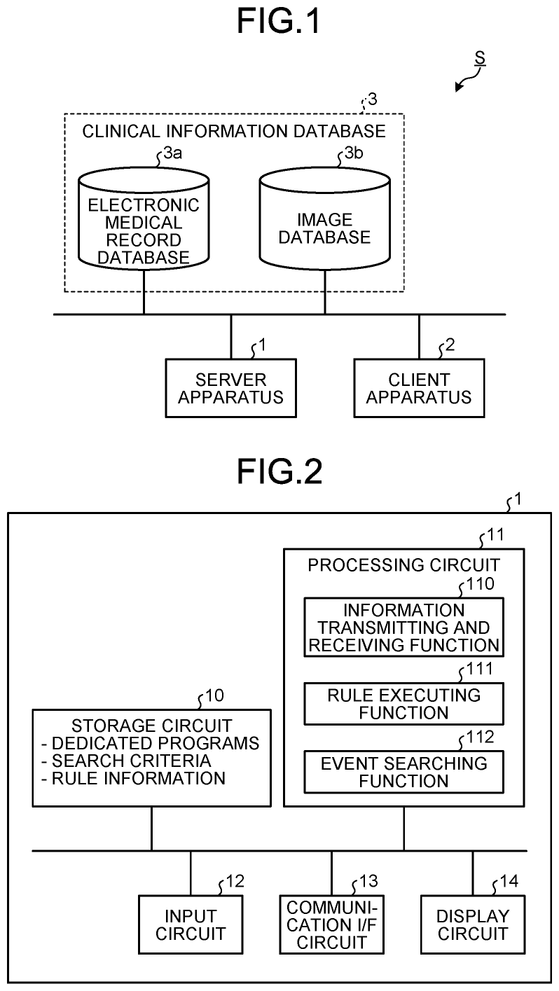

At first, an environment in which a medical information processing system and a medical information processing apparatus according to an embodiment are installed will be explained. FIG. 1 is a diagram illustrating a medical information processing system S including a server apparatus 1, a client apparatus 2, and a clinical information database 3. The medical information processing system S corresponds to the medical information processing system according to the embodiment. The server apparatus 1 and the client apparatus 2 each correspond to the medical information processing apparatus according to the embodiment.

In this situation, the server apparatus 1 and the clinical information database 3 do not necessarily have to be installed in a hospital and may be installed anywhere in an environment that allows communication with the client apparatus 2 via a network.

The clinical information database 3 includes an electronic medical record database 3a and an image database 3b. In this situation, the clinical information denotes information written in electronic medical records, information (e.g., nursing records, meal service information, medical accounting information) associated with electronic medical records, image data, and the like and is managed for each patient.

The electronic medical record database 3a is a database configured to store and manage, for each patient, the electronic medical records and the information associated with the electronic medical records. The electronic medical record database 3a according to the present embodiment may be a database serving as a Hospital Information System (HIS).

The image database 3b is a database configured to store and manage, for each patient, the image data obtained by various types of modalities and information associated with the image data. The image database 3b according to the present embodiment may be a database serving as a Picture Archiving and Communication System (PACS) and a Radiology Information System (RIS).

The server apparatus 1 is a dedicated-purpose or general-purpose computer. In response to a request from the client apparatus 2, the server apparatus 1 is configured to obtain corresponding clinical information from the clinical information database 3 and to transmit the obtained clinical information to the client apparatus 2. Further, the server apparatus 1 is configured to execute a rule, and when data matching the rule is present among the clinical information obtained from the clinical information database 3, to determine the matching clinical information as a subject of an alert. Further, the server apparatus 1 is configured to obtain, from the clinical information database 3, clinical information matching search criteria, by using the time of occurrence of the clinical information determined to be the subject of the alert (which hereinafter may be referred to as the “alert issuance time”), as a reference point in time.

In the present example, the “rule” denotes, in the situation where the clinical information is monitored to issue the alert (a warning), a rule defining which clinical information should be monitored and what standard should be used for issuing the alert. Typically, while a guideline defined for each disease is referenced, rules are determined in units of various elements such as hospitals, medical departments, doctors, patients, and the like.

Further, the “search criteria” denotes criteria used for searching, from among the information stored in the clinical information database 3, for a medical event from which the cause of the alert is analyzed. Each of the search criteria is a criterion used for searching for clinical information serving as a target of a search, from around the time at which the data matching the rule is present, typically a prescribed time period preceding the alert issuance time. The search criteria include at least the target of the search and a search time period and may be set for each rule and each patient, as necessary.

In this situation, the term “medical events” denotes all medical occurrences related to the patient and is represented by information managed for each patient. For example, medical events are represented by pieces of information such as “medication a was administered on MM/DD (month/day)”, “a CT image of the chest was taken on MM/DD”, and “an acute myocardial infarction on MM/DD”. The medical events are represented by information that is primarily specified by clinical information. Further, among the medical events, events that are subject to a search using one or more search criteria will be referred to as “searched events”.

In the present embodiment, clinical information for which a rule is executed will be referred to as “first clinical information”, whereas clinical information matching the search criteria will be referred to as a “second clinical information”.

The client apparatus 2 is, typically, a medical workstation installed in the hospital, a tablet computer used by a medical doctor, or the like. The client apparatus 2 is configured to display information related to treatments and medical examinations included electronic medical records and clinical information such as image information, so as to be integrated on a time axis.

Further, the client apparatus 2 is configured to request the server apparatus 1 to obtain, for example, specific clinical information serving as the first clinical information. Further, the client apparatus 2 is configured to display the second clinical information obtained according to the search criteria while using the alert issuance time as a reference point in time.

FIG. 2 is a diagram illustrating a configuration of the server apparatus 1 serving as a medical information processing apparatus.

The server apparatus 1 includes a storage circuit 10, a processing circuit 11, an input circuit 12, a communication interface (I/F) circuit 13, and a display circuit 14.

The storage circuit 10 is configured by using a semiconductor memory element such as a Random Access Memory (RAM) or a flash memory, or a hard disk, an optical disk, or the like. Alternatively, the storage circuit 10 may be configured by using a portable medium such as a Universal Serial Bus (USB) memory or a Digital Video Disk (DVD).

The storage circuit 10 has stored therein various types of processing programs (which may include application programs, an Operating System [OS], and the like) used by the processing circuit 11, as well as data necessary for executing the programs, volume data, and medical images. Further, the OS may include a Graphical User Interface (GUI) in which many graphical elements are used for causing the display circuit 14 to display information for an operator so as to make it possible to perform basic operations through the input circuit 12.

Further, the storage circuit 10 has stored therein information (rule information) related to a plurality of rules managed in units of hospitals, medical departments, medical doctors, patients, and the like.

Further, the storage circuit 10 has stored therein a plurality of search criteria managed in units of hospitals, medical departments, medical doctors, patients, and the like.

Next, the search criteria will be explained in detail. The search criteria are represented by information including at least a searched event and a search time period.

As a searched event, it is possible to set, for example, numerical value data related to vital signs or medication administration, character string data such as writings in medical records or nursing records, and the presence/absence of various types of medical events such as the presence/absence of surgery being performed.

With the numerical value data, it is possible to set a criterion indicating that a certain item exceeding a certain value or exceeding a specific change ratio. Further, for example, it is also possible to set a criterion using “AND” or “OR” with a plurality of items related to vital signs or the like, e.g., “the body temperature is 39° C. or higher AND the blood pressure is 170 mmHg” or “the body temperature is 39° C. or higher OR the blood pressure is 170 mmHg”.

With the character string data, it is possible to set a word or an arbitrary character string that serves as a search word. For example, it is possible to set a criterion using “AND” or “OR” with a plurality of search words, e.g., “a record having the word α AND the word β” or “a record having the word α OR the word β”.

With the presence/absence of medical events, it is possible to set various types of information such as the presence/absence of surgery being performed, the presence/absence of medication administration, as well as the presence/absence of a specific allergy, an infection, or a history of a disease.

The search time period denotes a prescribed time period which precedes the alert issuance time and in which the search is to be performed. For example, by setting the search time period as “within 12 hours preceding the alert issuance time”, it is possible to determine only such data that is estimated as being highly correlated with the occurrence cause of the data matching the rule, to be the target of the search.

As another example different from the above, it is also possible to set a search criterion so that “all the data within 2 hours from the alert issuance time used as a reference point in time shall be displayed as search results”.

Editing of the search criteria will be explained in detail later.

Returning to the description of FIG. 2, the processing circuit 11 is a processor configured to realize functions corresponding to the programs, by reading and executing the programs from the storage circuit 10. The processing circuit 11 includes, for example, an information transmitting and receiving function 110, a rule executing function 111, and an event searching function 112. By reading various types of control programs stored in the storage circuit 10, the processing circuit 11 is configured to realize the information transmitting and receiving function 110, the rule executing function 111, and the event searching function 112 and is also configured to integrally control processing operations of the storage circuit 10, the input circuit 12, the communication I/F circuit 13, and the display circuit 14. In other words, the processing circuit 11 that has read the programs has the functions illustrated within the processing circuit 11 in FIG. 2. The processing circuit 11 is an example of the first processing circuit.

The information transmitting and receiving function 110 is configured to transmit and receive various types of information including the clinical information to and from the client apparatus 2 and the clinical information database 3.

The rule executing function 111 is configured to execute the determined rule and to judge whether or not the clinical information being monitored matches the rule. When having determined that the clinical information being monitored matches the rule as a result of executing the rule, the rule executing function 111 is configured to determine the clinical information corresponding to the time when the rule is matched, as a subject for which an alert is to be issued.

By using the alert issuance time as a reference point in time, the event searching function 112 is configured to conduct a search in the clinical information database 3 according to the set search criteria and to obtain the second clinical information represented by a medical event relevant to the alert.

With reference to FIG. 2, the example was explained in which the single processor (i.e., the processing circuit 11) is configured to realize the rule executing function 111 and the event searching function 112; however, it is also acceptable to structure a processing circuit by combining together a plurality of independent processors, so that the functions are realized as a result of the processors executing the programs. Further, with reference to FIG. 2, the example was explained in which the single storage circuit (i.e., the storage circuit 10) has stored therein the programs corresponding to the processing functions; however, another arrangement is also acceptable in which a plurality of storage circuits 10 are provided in a distributed manner, so that the processing circuit 11 is configured to read a corresponding program from each of the individual storage circuits 10.

The term “processor” used in the above explanations denotes, for example, a Central Processing Unit (CPU), a Graphical Processing Unit (GPU), or a circuit such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]). One or more processors realize the functions by reading and executing the programs saved in the storage circuit 10. In this situation, instead of saving the programs in the storage circuit 10, it is also acceptable to directly incorporate the programs in the circuits of the one or more processors. In that situation, the one or more processors realize the functions by reading and executing the programs incorporated in the circuits thereof.

The input circuit 12 is a circuit configured to receive an input of a signal from an input device such as a pointing device (e.g., a mouse) and/or a keyboard that can be operated by the operator. In this situation, it is assumed that the input device itself is also included in the input circuit 12. When the input device is operated by the operator, the input circuit 12 is configured to generate an input signal corresponding to the operation and to output the generated signal to the processing circuit 11. In an example, the server apparatus 1 may include a touch panel in which the input device is integrally formed with the display circuit 14.

The input circuit 12 is realized by using a trackball, a switch button, a mouse, a keyboard, a touchpad on which input operations are performed by touching an operation surface thereof, a touchpad on which input operations are performed by touching an operation surface thereof, a touch screen in which a display screen and a touchpad are integrally formed, a contactless input circuit using an optical sensor, an audio input circuit, a touch panel display device in which a display screen and a touchpad are integrally formed, and/or the like, that are used for setting a region of interest (ROI), or the like.

The input circuit 12 does not necessarily have to include physical operation component parts such as a mouse, a keyboard, and/or the like. For instance, possible examples of the input circuit 12 include an electrical signal processing circuit configured to receive an electrical signal corresponding to an input operation from an external input device provided separately from the apparatus and to output the received electrical signal to a controlling circuit.

The communication interface (I/F) circuit 13 is configured to perform operations to communicate with external devices according to a predetermined communication standard. When the server apparatus 1 is provided on a network, the communication I/F circuit 13 is configured to transmit and receive information to and from external devices provided on the network. For example, the communication I/F circuit 13 is configured to receive data acquired in an imaging process from a medical image diagnosis apparatus such as a Magnetic Resonance Imaging (MRI) apparatus or a medical image managing apparatus.

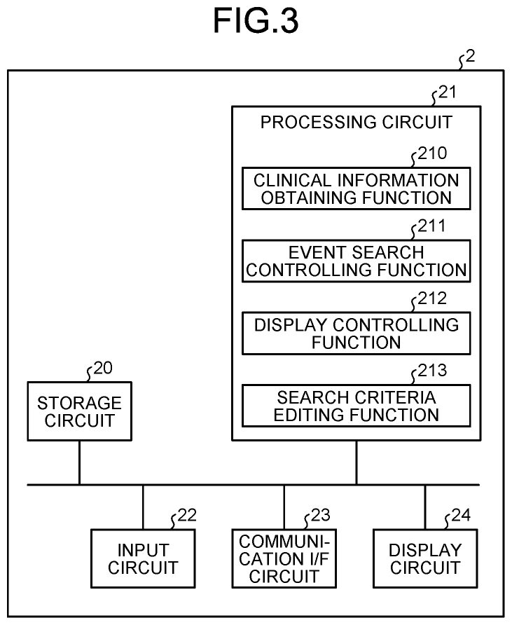

The display circuit 14 is a display device configured to display images and is configured by using a Liquid Crystal Display (LCD) monitor or the like. In response to an instruction from the processing circuit 11, the display circuit 14 is configured to cause the LCD to display various types of operation screens and various types of display information such as image data. FIG. 3 is a diagram illustrating a configuration of the client apparatus 2 serving as a medical information processing apparatus.

The client apparatus 2 includes a storage circuit 20, a processing circuit 21, an input circuit 22, a communication I/F circuit 23, and a display circuit 24.

The storage circuit 20 has a hardware configuration similar to that of the storage circuit 10. Further, the storage circuit 20 has stored therein dedicated programs used for realizing functions of the processing circuit 21, the clinical information obtained from the server apparatus 1, and the like.

The processing circuit 21 has a hardware configuration similar to that of the processing circuit 11. Further, the processing circuit 21 is a processor configured to realize the functions corresponding to the programs by reading and executing the programs from the storage circuit 20. For example, the processing circuit 21 includes a clinical information obtaining function 210, an event search controlling function 211, a display controlling function 212, and a search criteria editing function 213. By reading various types of control programs stored in the storage circuit 20, the processing circuit 21 is configured to realize the clinical information obtaining function 210, the event search controlling function 211, the display controlling function 212, and the search criteria editing function 213 and is also configured to integrally control processing operations of the storage circuit 20, the input circuit 22, the communication I/F circuit 23, and the display circuit 24. In other words, the processing circuit 21 that has read the programs has the functions illustrated within the processing circuit 11 in FIG. 3. The processing circuit 21 is an example of the second processing circuit.

The clinical information obtaining function 210 is configured to request and obtain the first clinical information for each patient from the server apparatus 1. Further, the clinical information obtaining function 210 is configured to obtain the second clinical information which was obtained according to the search criteria and uses the alert issuance time as a reference point in time, from the server apparatus 1.

With respect to the clinical information obtained according to the search criteria while using the displayed alert issuance time as a reference point in time, the event search controlling function 211 is configured to obtain linked information in response to an instruction from a user and to cause the display circuit 24 to display the linked information.

The display controlling function 212 is configured to cause the display circuit 24 to display the first clinical information obtained from the server apparatus 1. When there is first clinical information determined as a subject of the alert, the display controlling function 212 is configured to display the first clinical information with an alert. The display controlling function 212 is configured to display the obtained second clinical information on the basis of a designated priority order. The display controlling function 212 is configured to cause the display circuit 24 to display a search criteria setting screen used for setting search criteria.

The search criteria editing function 213 is configured to edit the search criteria on the basis of content that is input via the input circuit 22 on the search criteria setting screen displayed by the display circuit 24. In the present embodiment, the editing includes setting and changing processes. Further, in response to a save instruction, the search criteria editing function 213 is configured to transmit the edited search criteria to the server apparatus 1.

The input circuit 22, the communication I/F circuit 23, and the display circuit 24 have configurations similar to those of the input circuit 12, the communication I/F circuit 13, and the display circuit 14, respectively.

Next, a process (hereinafter, simply “second clinical information obtaining process”) to obtain the second clinical information represented by the medical event relevant to the alert according to the present embodiment will be explained.

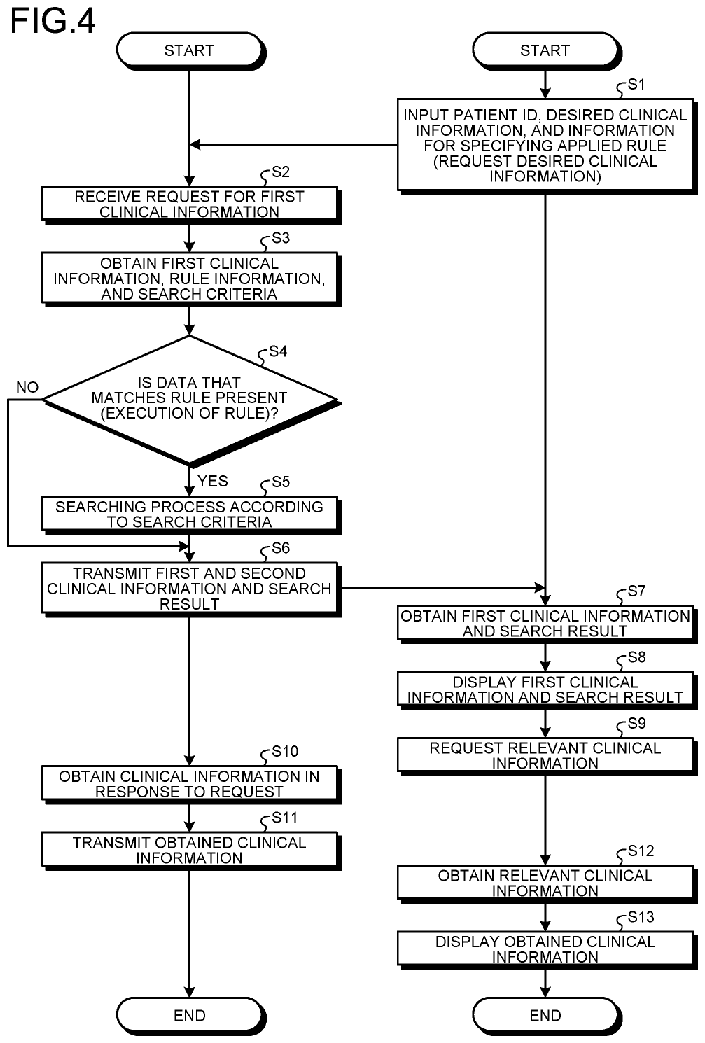

FIG. 4 is a flowchart illustrating a flow in the second clinical information obtaining process performed in the medical information processing system S.

As illustrated in FIG. 4, the clinical information obtaining function 210 of the client apparatus 2 receives inputs of a user login, a patient ID, an instruction to request a desired piece of clinical information (i.e., the first clinical information), and information for specifying an applied rule. The clinical information obtaining function 210 transmits the patient ID and the clinical information request instruction that were input, to the server apparatus 1 (step S1).

To explain the present embodiment specifically, let us discuss an example in which Blood Urea Nitrogen (BUN), C-Reactive Protein (CRP), and serum albumin (ALB) values have been input as the first clinical information. Further, as for the information for specifying the applied rule, for example, the information may automatically be input when a patient ID or clinical information is input.

The information transmitting and receiving function 110 of the server apparatus 1 receives the patient ID and the clinical information request received from the client apparatus 2 (step S2). According to the patient ID and the clinical information request that were received, the rule executing function 111 of the server apparatus 1 obtains the first clinical information of the patient, the rule associated with the patient, and the search criteria from the clinical information database 3. Further, according to the patient ID and the clinical information request that were received, the rule executing function 111 of the server apparatus 1 obtains the rule information and the search criteria associated with the patient, from the storage circuit 10 (step S3).

The rule executing function 111 of the server apparatus 1 executes the rule on the obtained clinical information of the patient and judges whether or not data matching the rule is present (step S4).

When the rule executing function 111 determines that data matching the rule is present (step S4: Yes), the event searching function 112 of the server apparatus 1 performs a searching process according to the search criteria (step S5). Accordingly, the event searching function 112 of the server apparatus 1 obtains second clinical information matching the search criteria from the clinical information database 3, by using the alert issuance time as a reference point in time.

The information transmitting and receiving function 110 of the server apparatus 1 transmits the first clinical information obtained at step S3 and the second clinical information obtained in the searching process at step S5, to the client apparatus 2 (step S6). On the contrary, when the rule executing function 111 determines that no data matching the rule is present (step S4: No), the information transmitting and receiving function 110 of the server apparatus 1 transmits the first clinical information obtained at step S3 to the client apparatus 2 (step 6).

The clinical information obtaining function 210 of the client apparatus 2 obtains the first clinical information and the second clinical information transmitted from the server apparatus 1 (step S7).

The display controlling function 212 causes the display circuit 24 to display the first clinical information and the second clinical information obtained at step S7 (step S8).

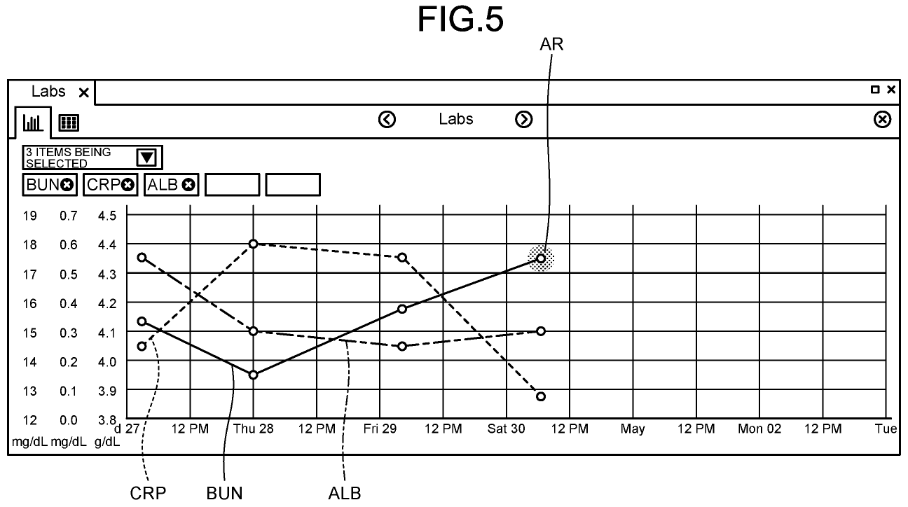

FIG. 5 is a drawing illustrating an example in which the first clinical information is displayed by the display circuit 24. As illustrated in FIG. 5, with respect to the patient, the values of BUN, CRP, and ALB on different days are displayed as being chronologically plotted. Further, as a result of executing the rule at step S4, the BUN value on Saturday, Jun. 30, 2019 is determined as data matching the rule and is displayed with an alert as indicated with the reference characters “AR”.

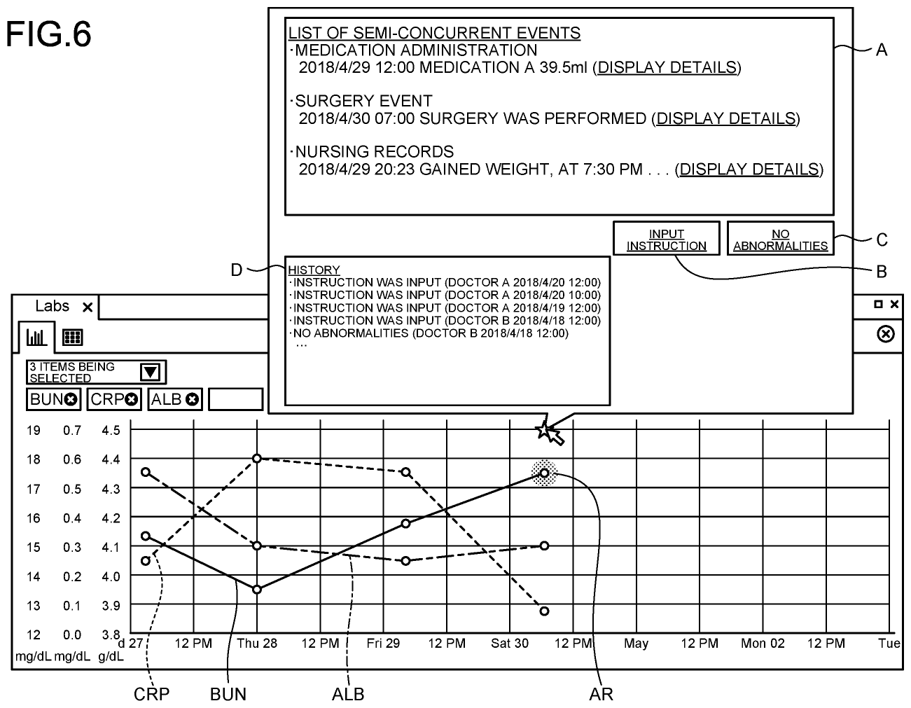

FIG. 6 is a drawing illustrating an example in which the second clinical information is displayed by the display circuit 24. As illustrated in FIG. 6, a marker is arranged with the time corresponding to the data displayed with the alert so that, when a mouse cursor is placed on the marker, the second clinical information represented by an event search result is displayed in a balloon.

In the example in FIG. 6, the second clinical information includes four pieces of content, namely, a list of events around the time in question (hereinafter “list of semi-concurrent events”) A, an “INSTRUCTION” button B, a “NO ABNORMALITIES” button C, and a history D.

In the list of semi-concurrent events A, the clinical information matching the searched event is displayed so that it is possible to understand, at a glance, “which data from what date/time matched”. In addition, displayed next to each of the pieces of information in the list of semi-concurrent events A is a “DISPLAY DETAILS” button. When an operation is performed on any of the “DISPLAY DETAILS” buttons, the display controlling function 212 causes the display circuit 24 to start up a new screen so as to display data being centered on the date/time of the corresponding data. Alternatively, the data may simultaneously be displayed in the same graph. With these arrangements, it is possible to easily make comparison of the data from around the alerted point, which reduces trouble in the analysis.

The “INSTRUCTION” button B and the “NO ABNORMALITIES” button C are provided. The user views the list of semi-concurrent events and if it is necessary to address an issue, the “INPUT INSTRUCTION” B button is selected, so as to take the next action such as instructing administration of a medication. When there are no abnormalities, the “NO ABNORMALITIES” button C is selected. In this situation, the selected result is saved.

The history D is displayed. The history indicates what actions were taken with respect to the searched event. Alternatively, another arrangement is acceptable in which, when the history D is selected, the display automatically skips to the data at the corresponding time. With these arrangements, by viewing the data from the past, it is possible to understand how the medical examination values changed as a result of the actions taken in the past. It is therefore possible to use the information as a reference for future actions.

After that, in response to the second clinical information displayed by the display circuit 24, when a request for further clinical information (third clinical information) is input by operating on a “DISPLAY DETAILS” button or the like, for example, the clinical information obtaining function 210 transmits a request for the corresponding clinical information to the server apparatus 1 (step S9).

The event searching function 112 of the server apparatus 1 obtains the requested clinical information from the clinical information database 3 (step S10). The information transmitting and receiving function 110 of the server apparatus 1 transmits the clinical information obtained by the event searching function 112 to the client apparatus 2 (step S1).

The clinical information obtaining function 210 of the client apparatus 2 obtains the transmitted clinical information (step S12). The display controlling function 212 of the client apparatus 2 causes the display circuit 24 to display, in a separate window for example, the clinical information obtained by the clinical information obtaining function 210 (step S13).

The processes at steps S9 through S13 are repeatedly performed multiple times as necessary.

### Search Criteria Editing Function

Next, a search criteria editing function according to the present embodiment will be explained. This function is to edit the search criteria used for obtaining the second clinical information from the clinical information database 3 while using the alert issuance time as a reference point in time.

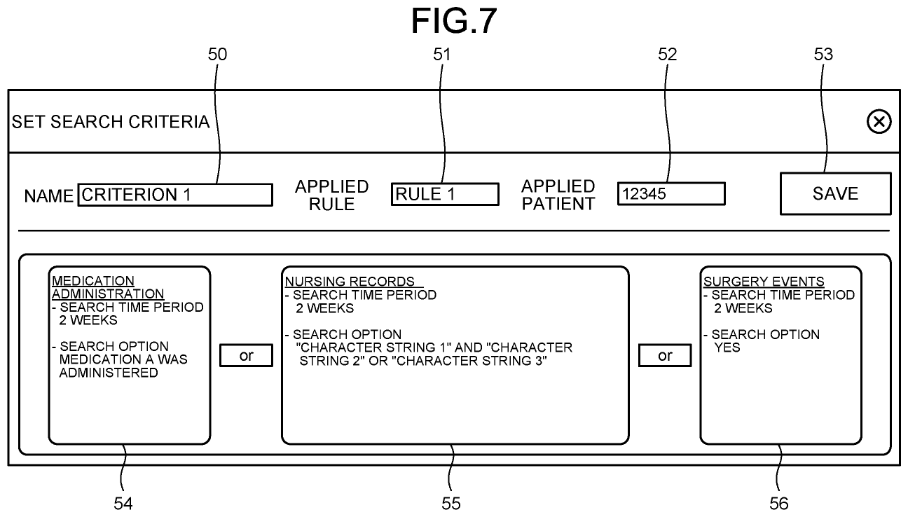

FIG. 7 is a drawing illustrating an example of a search criteria editing screen.

As illustrated in FIG. 7, the search criteria editing screen includes a name box 50, an applied rule box 51, an applied patient box 52, a “SAVE” button 53, applied event boxes 54, 55, and 56. By inputting necessary information to the boxes through the input circuit 22, it is possible to edit desired search criteria.

In the name box 50, a name (an ID) for identifying the search criteria is input.

In the applied rule box 51, information identifying a rule to be applied is input, in the situation where the search criteria are set to be used when data matching a specific rule is present. When the applied rule box 51 is empty, an event search is conducted with respect to all the rules.

In the applied patient box 52, information (the patient's name or ID) identifying an applied patient is input, in the situation where the search criteria are set to be used with the specific patient. When the applied patient box 52 is empty, an event search is conducted with respect to all the patients.

The “SAVE” button 53 is a button used for saving the edited search criteria. When the “SAVE” button 53 is pressed, the edited search criteria are saved in the storage circuit 20 and are also transmitted to the server apparatus 1 to be saved in the storage circuit 10.

In each of the applied event boxes, a search time period, a search option, and/or the like are input, for each of the medical events serving as a target of the search.

In FIG. 7, in the first applied event box 54, settings are made with respect to medical events of “medication administration”, while the search time period is set to the two weeks preceding the alert issuance time, whereas searching for “medication A was administered” is used as a search option.

Further, in the second applied event box 55, settings are made with respect to medical events of “nursing records”, while the search time period is set to the two weeks preceding the alert issuance time, whereas matching “[character string 1] and [character string 2] or [character string 3]” is used as a search option.

In the third applied event box 56, settings are made with respect to medical events of “surgery events”, while the search time period is set to the two weeks preceding the alert issuance time, whereas matching “YES” is used as a search option.

In the example in FIG. 7, the criteria in the applied event boxes 54, 55, and 56 are set with “ORs”. Alternatively, it is also possible to set the criteria with “ANDs” or to set the criteria with a combination of an “AND” and an “OR”.

Further, although FIG. 7 illustrates the example using the three applied event boxes, there is no limitation to the number of applied event boxes. The user is able to freely increase or decrease the number of applied event boxes by performing a predetermined operation.

By using the search criteria editing function described above, the user is able to change the search criteria to have arbitrary content and to add one or more new search criteria, at an arbitrary time.

In the present embodiment, the client apparatus 2 serving as a medical information processing apparatus includes the search criteria editing function 213. Alternatively, the server apparatus 1 serving as a medical information processing apparatus may include the search criteria editing function 213.

As explained above, the medical information processing system according to the present embodiment includes the clinical information database 3, the server apparatus 1, and the client apparatus 2. The server apparatus 1 includes the rule executing function 111 serving as the rule executing unit and the event searching function 112 serving as a searching unit. The rule executing function 111 is configured to judge whether or not first clinical information matching the rule is present and to issue an alert when it is determined that the first clinical information matching the rule is present. The event searching function 112 is configured to conduct a search in the clinical information database 3 according to the search criteria, from during the time period preceding the reference point in time at which the first clinical information matching the rule occurred, so as to obtain the second clinical information relevant to the first clinical information, and to transmit the obtained second clinical information to the client apparatus 2. The client apparatus 2 includes the clinical information obtaining function 210 serving as an obtaining unit and the display controlling function 212 serving as a display controlling unit. The clinical information obtaining function 210 is configured to obtain the transmitted second clinical information. The display controlling function 212 is configured to cause the display circuit 24 to display the obtained second clinical information.

Accordingly, from during the prescribed time period preceding the alert issuance time, it is possible to automatically extract and provide the second clinical information relevant to the first clinical information. By using the provided second clinical information, the user is able to perform the data analyses properly.

As a result, it is possible to reduce the trouble of searching for the data in the prescribed time period preceding the alert issuance time and to thus improve efficiency in diagnosis/treatment processes. In the situation where an alert is issued as a result of monitoring specific clinical information according to a rule, it is possible to reduce the work burden imposed on the user at the time of understanding the cause of the alert issuance. Further, in the medical field, reducing this type of trouble will enhance the quality of medical care and will be able to prevent oversights that may be caused by human-based methods.

### First Modification Example

In the above embodiment, when details are displayed for the list of semi-concurrent events included in the second clinical information, for example, the detailed information may be too much to be displayed all at once in some situations. In those situations, by pressing the “AUTOMATIC PANEL LAYOUT button”, it is possible to have a layout determined automatically.

For example, at the time of setting a searched event, it is possible to set priority levels for displaying the clinical information.

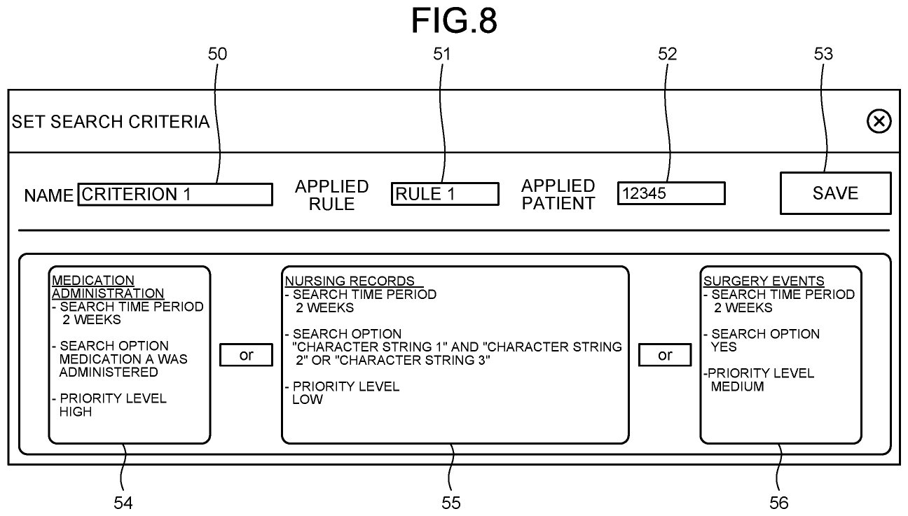

FIG. 8 is a drawing illustrating an example of a screen used for setting priority levels for displaying the clinical information. As illustrated in FIG. 8, in the applied event boxes 54, 55, and 56, it is possible to set the display priority levels. In the example in FIG. 8, the medication administration is set with a “priority level: high”; the nursing records are set with a “priority level: low”; and the surgery is set with a “priority level: medium”.

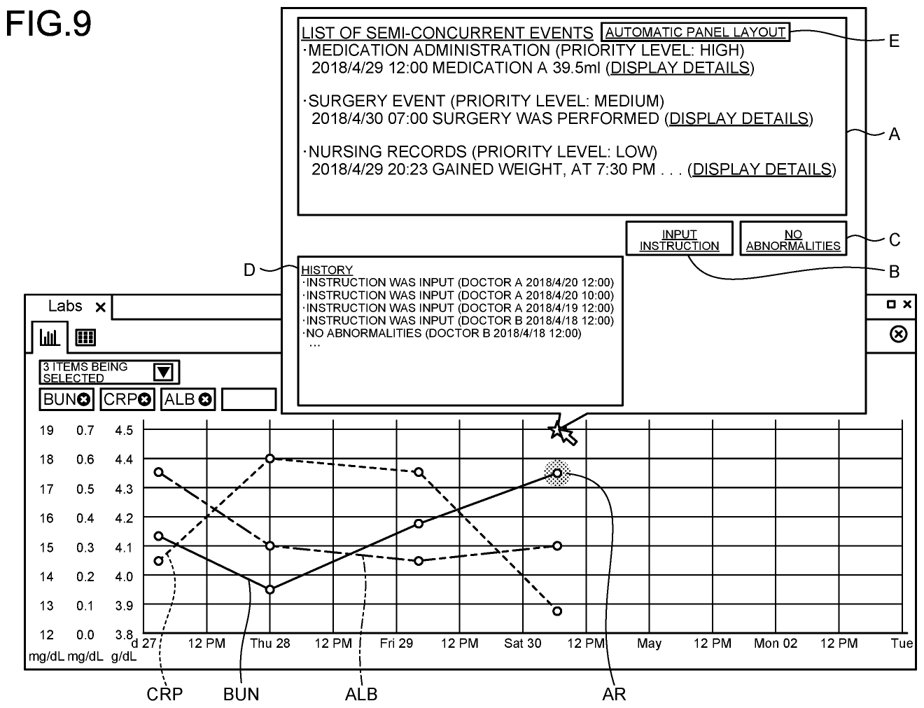

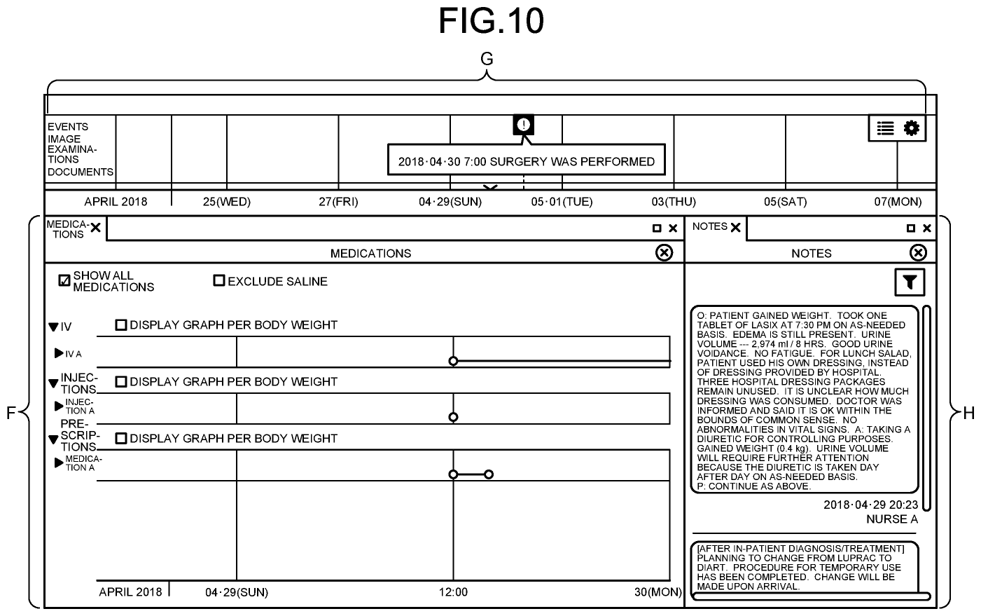

FIGS. 9 and 10 are drawings for explaining examples of screens used for displaying the second clinical information when it is possible to set display priority levels.

As illustrated in FIG. 9, in the list of semi-concurrent events A, the medication administration set with the “priority level: high”, the surgery event set with the “priority level: medium”, and the nursing record set with the “priority level: low” are displayed, according to the settings of the display priority levels of the clinical information illustrated in FIG. 8.

Further, as illustrated in FIG. 9, in the list of semi-concurrent events A, the “AUTOMATIC PANEL LAYOUT button” is provided. When the “AUTOMATIC PANEL LAYOUT button” is pressed, the display controlling function 212 automatically lays out the panels (windows) used for displaying the details, as illustrated in FIG. 10, according to the settings of the display priority levels of the clinical information illustrated in FIG. 8. Each of the panels displays the second diagnosis/treatment information represented by an event search result. The display controlling function 212 is configured to control the size of the panels on the basis of the display priority levels in the settings.

In the example in FIG. 10, a panel F corresponding to the medication administration event set with the “priority: high” is arranged in the center, while another panel G corresponding to the surgery event set with the “priority: medium” is arranged at the top, and yet another panel H corresponding to the nursing record set with the “priority: low” is arranged on the right.

Alternatively, another arrangement is also acceptable in which the area sizes in the panel layout are determined according to the priority levels only at the beginning, so that if the user subsequently changes the area size of any of the panels after the panels are automatically laid out, the area sizes are corrected in the manner of feedback, on the basis of a result of the change.

The above configuration makes it possible to browse the details of the semi-concurrent events more easily and saves the user from the trouble of adjusting the positions of the panels.

### Second Modification Example

In the above embodiment, the BUN, CRP, ALB values obtained from blood tests were used as examples of the first clinical information. Alternatively, another arrangement is also acceptable in which, for example, the server apparatus 1 is configured to continuously obtain blood pressure values or the like from a wearable terminal so as to monitor the values in a real-time manner and to issue an emergency alert for the client apparatus 2 at a point in time when a rule is matched.

### Third Modification Example

In the above embodiment, the example was explained in which the server apparatus 1 performs the searching process according to the rule and the search criteria after receiving the request from the client apparatus 2. Alternatively, another arrangement is also acceptable in which, the server apparatus 1 follows a setting and automatically performs the searching process according to the rule and the search criteria, before receiving the request from the client apparatus 2. Further, the searching process according to the search criteria does not necessarily have to be started automatically and may be triggered by a start instruction given by the user through the input circuit 22.

According to at least one aspect of the embodiments described above, when the alert is issued as a result of monitoring specific clinical information according to the rule, it is possible to reduce the work burden imposed on the user at the time of understanding the cause of the alert issuance.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising processing circuitry configured:
to judge whether or not first clinical information matching an alert issuance reference rule is present;
to issue an alert when having determined that the first clinical information matching the alert issuance reference rule is present;
to obtain second clinical information relevant to the first clinical information from during a time period preceding a reference point in time at which the first clinical information matching the alert issuance reference rule occurred; and
to cause display circuitry to display the second clinical information.

2. The medical information processing apparatus according to claim 1, wherein the second clinical information includes at least one of the following: a medical event relevant to the first clinical information matching the alert issuance reference rule; an instruction from a medical doctor in response to the alert; a determination result indicating absence of abnormalities in response to the alert; and a history of addressing a past alert issuance regarding the first clinical information.

3. The medical information processing apparatus according to claim 1, further comprising: input circuitry configured to input a requesting instruction to request third clinical information associated with the second clinical information, wherein
the processing circuitry causes the display circuitry to display the third clinical information in response to the requesting instruction from the input circuitry.

4. The medical information processing apparatus according to claim 1, wherein the processing circuitry is configured to conduct a search in the clinical information database according to a search criterion so as to obtain the second clinical information relevant to the first clinical information.

5. The medical information processing apparatus according to claim 4, wherein
the search criterion includes at least a medical event being a target of the search and a search time period, and
the second clinical information includes a medical event being the target of the search corresponding to the search time period.

6. The medical information processing apparatus according to claim 4, wherein the second processing circuitry is configured to edit the search criterion.

7. The medical information processing apparatus according to claim 6, wherein
the search criterion includes at least one of numerical value data, character string data, and information indicating presence/absence of a medical event, and
the second processing circuitry edits the numerical value data, the character string data, and the information indicating the presence/absence of the medical event.

8. The medical information processing apparatus according to claim 1, wherein the second clinical information includes at least one of the following: the medical event relevant to the first clinical information matching the alert issuance reference rule; input circuitry used for inputting an instruction of a medical doctor in response to the alert; input circuitry used for inputting a determination result indicating absence of abnormalities in response to the alert; and a history of addressing a past alert issuance regarding the first clinical information.

9. A medical information processing system comprising a clinical information database, a server apparatus, and a client apparatus, wherein
the server apparatus includes first processing circuitry,
the first processing circuitry is configured:
to judge whether or not first clinical information matching an alert issuance reference rule is present; to issue an alert when having determined that the first clinical information matching the alert issuance reference rule is present; and
to conduct a search in the clinical information database according to a search criterion, from during a time period preceding a reference point in time at which the first clinical information matching the alert issuance reference rule occurred, so as to obtain second clinical information relevant to the first clinical information, and to transmit the obtained second clinical information to the client apparatus,
the client apparatus includes second processing circuitry, and
the second processing circuitry is configured:
to obtain the transmitted second clinical information; and
to cause display circuitry to display the obtained second clinical information.

10. The medical information processing system according to claim 9, wherein
the search criterion includes at least a medical event being a target of the search and a search time period, and
the second clinical information includes a medical event being the target of the search corresponding to the search time period.

11. The medical information processing system according to claim 10, wherein the second clinical information includes at least one of the following: the medical event relevant to the first clinical information matching the alert issuance reference rule; input circuitry used for inputting an instruction of a medical doctor in response to the alert; input circuitry used for inputting a determination result indicating absence of abnormalities in response to the alert; and a history of addressing a past alert issuance regarding the first clinical information.

12. The medical information processing system according to claim 9, wherein
the client apparatus further includes input circuitry configured to input a request instruction for third clinical information associated with the displayed second clinical information,
the second processing circuitry obtains the third clinical information from the clinical information database in response to the request instruction from the input circuitry, and
the second processing circuitry causes the display circuitry to display the obtained third clinical information.

13. The medical information processing system according to claim 12, wherein, when two or more pieces of third clinical information are present, the second processing circuitry causes the display circuitry to display the two or more pieces of third clinical information on a basis of priority levels related to the display.

14. The medical information processing system according to claim 9, wherein the second processing circuitry is configured to edit the search criterion.

15. The medical information processing system according to claim 14, wherein
the search criterion includes at least one of numerical value data, character string data, and information indicating presence/absence of a medical event, and
the second processing circuitry edits the numerical value data, the character string data, and the information indicating the presence/absence of the medical event.

16. The medical information processing system according to claim 9, wherein the second clinical information includes at least one of the following: a medical event relevant to the first clinical information matching the alert issuance reference rule; an instruction from a medical doctor in response to the alert; a determination result indicating absence of abnormalities in response to the alert; and a history of addressing a past alert issuance regarding the first clinical information.

17. The medical information processing system according to claim 9, further comprising: input circuitry configured to input a requesting instruction to request third clinical information associated with the second clinical information, wherein
the processing circuitry causes the display circuitry to display the third clinical information in response to the requesting instruction from the input circuitry.

18. A medical information processing apparatus comprising processing circuitry configured:
to obtain second clinical information from a clinical information database according to a search criterion, when an alert is issued as a result of an alert issuance reference rule executing circuitry determining that first clinical information matching an alert issuance reference rule is present, the obtained second clinical information being relevant to the first clinical information during a time period preceding a reference point in time at which the first clinical information matching the alert issuance reference rule occurred; and
to cause display circuitry to display the obtained second clinical information.

19. A medical information processing method comprising:
judging whether or not first clinical information matching an alert issuance reference rule is present;
issuing an alert when it is determined that the first clinical information matching the alert issuance reference rule is present;
obtaining second clinical information from a clinical information database according to a search criterion, the obtained second clinical information being relevant to the first clinical information during a time period preceding a reference point in time at which the first clinical information matching the alert issuance reference rule occurred; and
causing display circuitry to display the obtained second clinical information.
